(19) United States
(12) Plant Patent Application Publication
(10) Pub. No.: US 2016/0050828 P1
(43) Pub. Date: Feb. 18, 2016

(54) Clematis plant named 'Evipo062'

(50) ;

(71) Applicants: Mogens Nyegaard Olesen, Fredensborg (DK); Raymond Evison, St. Sampsons (GB)

(72) Inventors: Mogens Nyegaard Olesen, Fredensborg (DK); Raymond Evison, St. Sampsons (GB)

(21) Appl. No.: 14/121,277

(22) Filed: Aug. 15, 2014

(51) Int. Cl. A01H 5/00

(52) U.S. Cl. PLT/228

(57) ABSTRACT

A new Clematis plant with a compact growth habit, profuse, blue flowers, and continuous summer flowering. The variety successfully propagates from softwood cuttings and is suitable for cultivation commercial nursery culture. This new and distinct variety has shown to be uniform and stable in the resulting generations from asexual propagation from vegetative cuttings.

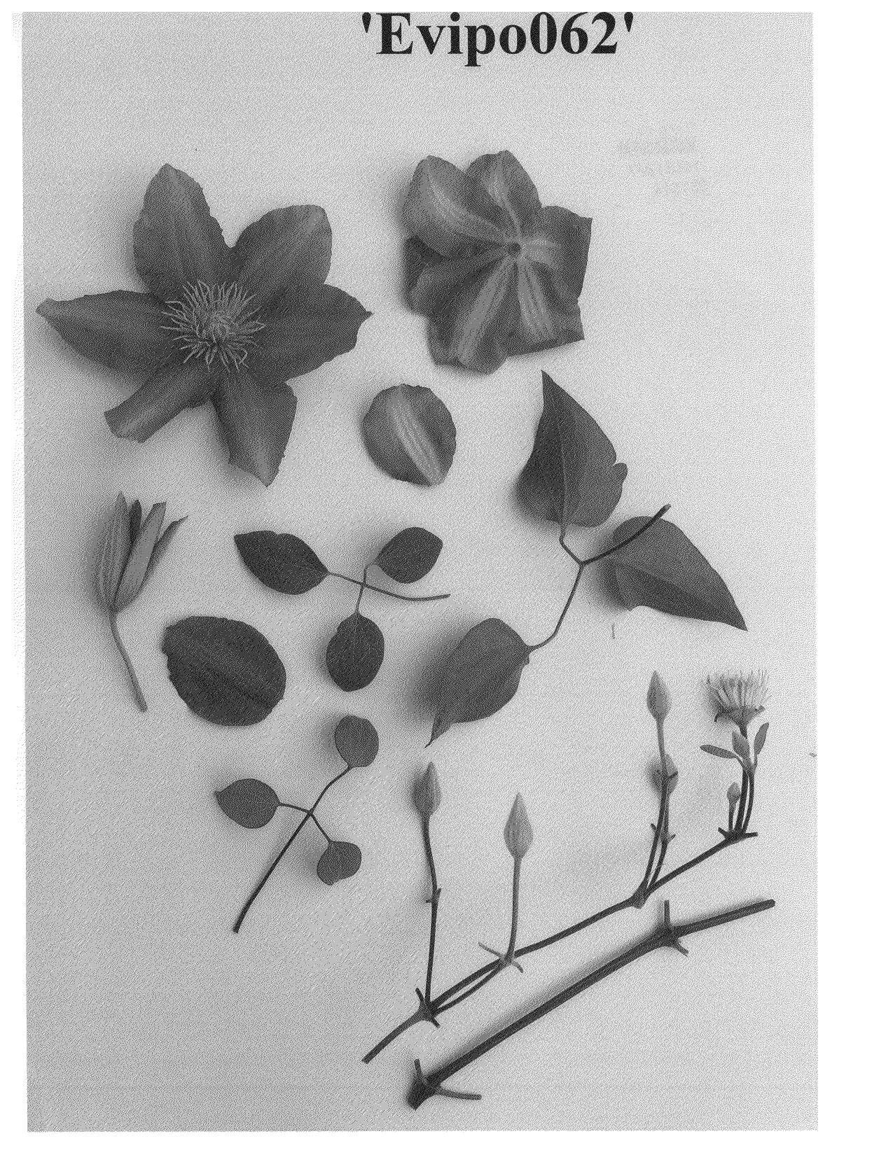

### BOTANICAL CLASSIFICATION

Genus: Clematis

Species: viticella

### VARIETY DENOMINATION

‘Evipo062’

### SUMMARY OF THE CLAIMED PLANT

The present invention constitutes a new and distinct variety of Clematis plant which originated from a controlled crossing between the female seed parent, an unnamed seedling, and the male pollen parent, an un-named seedling

The two parents were crossed during the summer of 2000 and the resulting seeds were planted the following winter in a controlled environment in Guernsey, Channel Islands. The new variety named ‘Evipo062’ originated as a single seedling from the stated cross.

The new Clematis plant may be distinguished from its female seed parent and male pollen parent by the following characteristics.

The objective of the hybridization of this Clematis plant was to create a new and distinct variety for nursery culture with unique qualities such as:

1. Uniform and abundant blue flowers;

2. Vigorous and compact growth, making the variety suitable for container culture; and

3. Improved disease resistance.

This combination of qualities was lacking in Clematis plants that were in commercial cultivation and the qualities have been substantially achieved in the new variety.

‘Evipo062’ was selected by Mogens N. Olesen and Raymond J. Evison in their Clematis development program in the Channel Islands, United Kingdom in 2001. Asexual reproduction of ‘Evipo062’ by means of vegetative cuttings and traditional layering was first performed by Mogens N. Olesen and Raymond J. Evison in the nursery during the summer of 2001. This initial and subsequent asexual propagations have demonstrated that the characteristics of ‘Evipo062’ are true to type and are transmitted from one generation to the next.

### BRIEF DESCRIPTION OF THE DRAWING

The accompanying color illustration shows as true as is reasonably possible to obtain in color photographs of this type the typical characteristics of the buds, flowers, leaves, and stems, of ‘Evipo062’. Specifically illustrated in the drawing are flowers at various stages of development, flower in parts, leaves, and stems. Plants in the photographs are 2 years of age.

### DETAILED DESCRIPTION OF THE VARIETY

The following is a detailed description of ‘Evipo062’, as observed in its growth throughout the flowering period in open air cultivation in Yamhill County Oreg. Observed plants were cultivated for a period of 18 months in 2 liter containers. Certain phenotypical characteristics of the variety may vary under different environmental, cultural, agronomic, seasonal, and climatic conditions. Color references are made using The Royal Horticultural Society (London, England) Colour Chart, 2001, except where common terms of color are used.

For a comparison, several physical characteristics of the Clematis variety ‘Evipo023’ described and illustrated in U.S. Plant Pat. No. 15,166 issued Sep. 21, 2004 are compared to ‘Evipo062’ in Chart 1.

CHART 1

| | ‘Evipo062’ | ‘Evipo023’ |
|---|---|---|
| Flower diameter | 110 mm | 90 to 120 mm |
| Tepal upper | Violet-Blue Group | RHS 91A of the Violet Blue |
| surface after | 94B | Group |
| opening | | |
| Tepal count | 6 | 6-8 |

Flower and flower bud:
   Blooming habit.—Recurrent. The natural flowering period is generally from June to September.
   Flower bud.—Size: Normally 29 mm in length. Bud diameter is 14 mm. Bud form: Elliptic. Bud color: Yellow-Green Group 145C.
   Peduncle.—Surface Texture: Smooth. Length: On average 40 mm. Color: Yellow-Green Group 145A and Greyed-Purple Group 183A. Strength: Strong.
   Receptacle.—Surface Texture: Smooth. Shape: Funnel. Size: 1 mm (h)×2 mm (w). Color: Yellow-Green Group 145A.
   Flower arrangement.—Location on vine: New growth. Borne: Normally in clusters of 5 to 7 flowers per flowering branch. Flowers initiate from terminal and axillary buds. Aspect: Flowers are oriented upwards and to 45 degree angle.
   Flower bloom.—Size: On average, flowers are 110 mm in diameter and 20 mm in depth. Fragrance: None. Lasting Quality: Flowers normally remain 7 days on the plant.
   Tepals.—Tepal Color: The upper surface is Violet-Blue Group 94B. The lower surface is Violet-Blue Group 94C with a central bar of Yellow-Green Group 145D. Quantity: Normally 6 tepals. Size: 55 mm in length by 35 mm wide. Shape: Individual tepal shape is elliptic. The tepal apex is acute. The tepal base is acute. Apex Recurvature: Slightly recurved. Tepal Cross section: Flat. Margins: Entire. Weak undulations of margin observed. Persistence: Tepals drop off cleanly/hang on and dry.
   Reproductive organs.—Arrangement: Open. Pollen: Not observed. Anthers: Size: 3 mm in length. Color: White Group 155A. Quantity: On average, 45. Filaments: Color: White Group N155A. Length: 6 mm. Pistils: Quantity: On average, 20. Stigmas: Inferior in location relative to the length of the filaments and the height of the anthers. Styles: Color: White Group 155A. Length: 5 mm.
   Seed head characteristics.—Not observed.
Plant:
   Plant form.—Climbing.
   Plant growth.—Moderately vigorous.
   Size.—Seasons growth attains 1 meter in height. Average spread is 50 cm.
   Hardiness.—Trials to date show the variety hardy in USDA Zones 4-9.
   Stems.—Color: Juvenile stems are Yellow-Green Group 144A with intonations of Greyed-Purple Group 183A. Mature stems are Greyed-Orange Group 166B. Internodes: 7 to 9 cm between nodes. Length: Normally 35 cm from the base of the plant to the flowering portion of the stem. Diameter: Normally 3 mm. Texture: Mature stems are ribbed.
   Plant foliage.—Leaf characteristics: Deciduous. Arrangement: Trifoliate. Leaf Size: Compound leaves are normally 80 mm (l)×70 mm (w). Leaflets are normally 35 mm (l)×20 mm (w). Abundance: On average 2 leaves per 10 cm of stem. Leaf Color: Juvenile upper Yellow-Green Group 144A. Juvenile lower Yellow-Green Group 144A. Mature upper Yellow-Green Group 146A. Mature lower Yellow-Green Group 146B. Stipules: Absent. Petioles: Size: Normally 35 mm in length by 1 mm diameter. Texture: Smooth. Color: Greyed-Purple Group 183A and Yellow-Green Group 144A. Petioloule: Size: Normally 10 mm in length by 1 mm diameter. Texture: Smooth. Color: Greyed-Purple Group 183A and Yellow-Green Group 144A. Leaflet Shape: Elliptic. Margin: Entire. Surface: The upper side is smooth. The lower side is smooth. Thickness: Moderate. Glossiness: Not glossy.
   Disease resistance.—Subject to any disease that normally attacks the species. However the variety is more tolerant to Clematis wilt, Phoma clematidina, than some Clematis.
   Cold hardiness.—The variety is tolerant to USDA Hardiness Zone 6.
   Heat tolerance.—The variety has been found to be suitable for climate conditions found in the American Horticulture Society heat zone 7.

We claim:

1. A new and distinct variety of Clematis plant named ‘Evipo062’, substantially as described and illustrated, due to its abundant blue flowers with good keepability, attractive long lasting foliage and compact growth, year round flowering under glasshouse conditions, suitability for production from softwood cuttings in pots, durable flowers and foliage which make the variety suitable for distribution in the floral industry.
